(19) United States
(12) Patent Application Publication
(10) Pub. No.: US 2005/0154284 A1
(43) Pub. Date: Jul. 14, 2005

(54) Method and system for calibration of a marker localization sensing array

(76) Inventors: J. Nelson Wright, Mercer Island, WA (US); Laurence J. Newell, Mercer Island, WA (US)

Correspondence Address: PERKINS COIE LLP;PATENT-SEA, P.O. BOX 1247, SEATTLE, WA, 98111-1247, US

(21) Appl. No.: 10/750,456

(22) Filed: Dec. 31, 2003

(51) Int. Cl. A61B 5/05

(52) U.S. Cl. 600/407

(57) ABSTRACT

A method and system for calibrating a sensing array used in marker localization. The sensing array is for sensing a signal produced by a marker is implanted in an object, such as a human body. The signal generated by the marker is a magnetic field. The sensing array has a plurality of sensing coils and associated amplification circuitry. The method comprises applying an excitation to each of the sensing elements and analyzing the output of the plurality of sensing elements resulting from the excitation. A correction matrix based upon the analyzed outputs of the plurality of sensing elements is determined.

### CROSS-REFERENCE TO RELATED APPLICATIONS

This application is related to U.S. patent application Ser. No. 10/334,700 filed Dec. 30, 2002, U.S. patent application Ser. No. 10/382,123, filed Mar. 4, 2003, and U.S. patent application Ser. No. 10/679,801 filed Oct. 6, 2003, all of which are incorporated herein by reference in their entirety.

### BACKGROUND

Implantable markers have been used to identify locations within objects, such as a human body. For example, a marker may be implanted in a patient within an organ of interest. As the patient moves, the marker can be used to track the location of the organ. Various techniques have been used to identify the location of such markers.

As described in my co-pending U.S. patent applications noted above, one technique for locating a marker is by measuring the magnetic flux generated by the marker upon excitation from a source. Thus, after excitation, the source excitation is shut down and an observation (listening) period begins. During the observation period, the resonant wireless marker emits a magnetic dipole field whose time-domain waveform is an exponentially decaying sinusoid. The observation period lasts roughly 48 cycles of the marker resonant frequency. There is no ‘accuracy’ requirement on the resonant marker construction. Its small size and construction guarantee a precise magnetic dipole field at relevant sensing distances.

The measurement of the magnetic flux is typically performed by an array of sensing elements that together form a sensing array. The sensing elements are typically sensing coils as described in our co-pending applications. The planar sensing array and associated receiver electronics sense, or measure, the dipole field generated by the resonant marker. The system thereby measures the magnetic flux captured by each of the many sense coils in the array.

The system determines a least-square-error estimate of the location of the marker by comparing the measured spatial pattern of received signals from the marker dipole field with a model of the reception of dipole signal fields by the array. Channel-to-channel gain variation, or electronic crosstalk present in the sense array or receiver electronics and not accounted for in the model can result in error in the estimate of the marker's location. Parasitic impedances in the sensing array circuitry not in the model will also contribute to localization estimate error. For these reasons, it is desirable to accurately account for these and other effects, such as by use of a calibration method and apparatus.

### BRIEF DESCRIPTION OF THE DRAWINGS

FIG. 1 is a perspective view of an example of a system for estimating the location of wireless implantable markers.

FIG. 2 is a block diagram illustrating components of the system of FIG. 1 including a sensing subsystem.

FIG. 3A is an exploded isometric view showing individual components of a sensing subsystem in accordance with an embodiment of the invention.

FIG. 3B is a top plan view of an example of a sensing assembly of a sensing subsystem.

FIG. 4 is a schematic diagram of a preamplifier adapted with calibration circuitry and for use with the sensing array.

FIG. 5 is an equivalent circuit for the preamplifier of FIG. 4.

FIG. 6 is an illustration of the model used for the calibration of the present invention.

FIG. 7 is a flow diagram illustrating the method of static calibration.

FIG. 8 is a flow diagram illustrating the method of dynamic calibration.

FIG. 9 is a flow diagram illustrating the calibration process of the present invention.

Sizes of various depicted elements are not necessarily drawn to scale, and these various elements may be arbitrarily enlarged to improve legibility. Also, the headings provided herein are for convenience only and do not necessarily affect the scope or meaning of the claimed invention.

### DETAILED DESCRIPTION

The subject of the present invention is a calibration and measurement architecture which accurately measures array/receiver electronic gain and crosstalk terms that affect the magnetic flux signals captured by the array from the marker. By accurately measuring these gain and crosstalk terms, their ‘inverse’ can be applied to the measured (corrupted) signals to recover the actual magnetic flux captured at each coil.

It is also important to note that the gain and crosstalk present in the array and receiver electronics can vary with time, temperature or system operating condition. The present invention describes a calibration architecture which is built-in to the system. The system performs calibration autonomously by executing calibration measurements interleaved with localization measurements, thereby measuring any variations in gain and crosstalk which vary with time, temperature or system operating condition.

Thus, the present invention provides a method for calibrating a sensing array used in implanted marker localization. In one embodiment, the marker emits magnetic flux and the sensing array measures the magnetic flux. While the description below is specifically directed to a sensing array for magnetic flux, the present invention can be applied to other types of sensing arrays that measure other phenomena.

As detailed below, the present invention measures channel-to-channel crosstalk as well as channel gain. By “channel”, it is meant the signal path from an individual sensing element or sense coil. In one embodiment, there are thirty-two sense coils in a sensing array and thus thirty-two channels. Each calibration measurement made by the system consists of exciting a single channel's calibration source, while measuring the response on all thirty-two channels, including the excited channel.

Further, the present invention measures system gain and crosstalk with a ‘surrogate’ calibration signal electrically equivalent to a Faraday voltage induced in the sense coil. This calibration voltage source is implemented by ‘driving’ the bases of the common-base PNP transistor amplifier. This effectively creates a voltage source in series with the sense coil, which is mathematically treated the same as a Faraday voltage induced in the sense coil from an externally generated AC magnetic flux (such as that from the resonant marker). While the ‘surrogate’ calibration signal is mathematically interchangeable with the Faraday voltage (induced by the resonant marker's AC magnetic field), there yet is a small correction needed. The calibration architecture provides a means for measuring a small correction needed to relate these two.

Implementation of the array calibration architecture is created from a minimum of active (semiconductor) parts. The sense array in normal use is exposed to a significant lifetime dose of high-energy photon radiation (˜40 krad). The present calibration and preamplifier architecture uses only a few discrete PNP transistors (which are fairly robust to radiation) and a few standard CMOS logic gates per channel (which can be purchased radiation hardened).

Accuracy of the calibration architecture is determined solely by the ability to provide matched resistors in all channels. Resistors are available with accuracies to 0.01% (100 ppm) and very low temperature coefficients. Temperature in most applications is well-controlled and temperature is well-matched across the sensing array.

The invention will now be described with respect to various embodiments. The following description provides specific details for a thorough understanding of, and enabling description for, these embodiments of the invention. However, one skilled in the art will understand that the invention may be practiced without these details. In other instances, well-known structures and functions have not been shown or described in detail to avoid unnecessarily obscuring the description of the embodiments of the invention.

Description of Suitable Systems

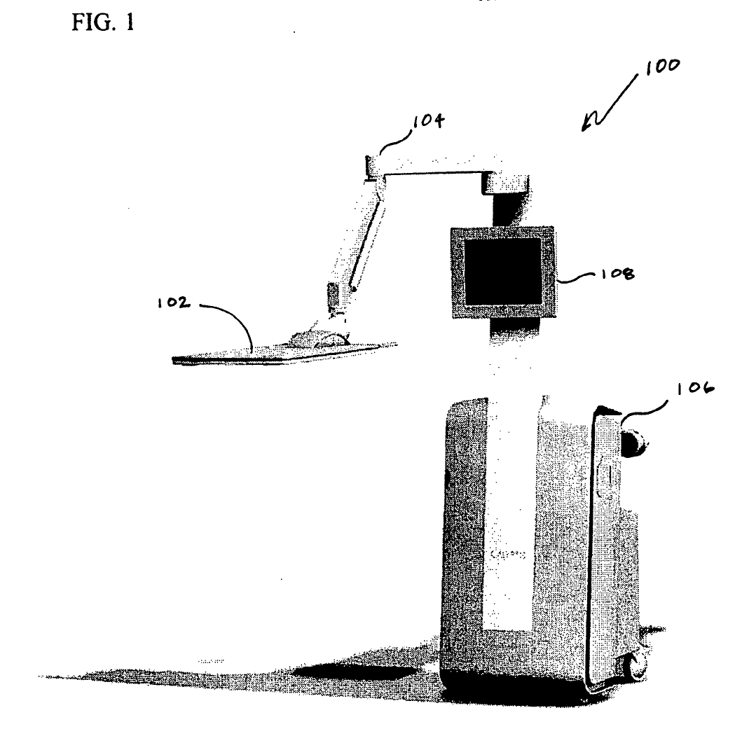

FIG. 1 is a perspective view showing an example of a system 100 for energizing and locating one or more wireless markers in three-dimensional space. The system includes an excitation source and sensor array 102 supported by a movable arm 104. The arm 104 is secured to a base unit 106 that includes various components, such as a power supply, computer (such as an industrial personal computer), and input and output devices, such as a display 108. Many of these components are described in detail below.

The system 100 may be used with guided radiation therapy to accurately locate and track a target in a body to which guided radiation therapy is delivered. Further details on use of the system with such therapy may be found in U.S. patent application Ser. No. 09/877,498, entitled “Guided Radiation Therapy System,” filed Jun. 8, 2001, which is herein incorporated by reference.

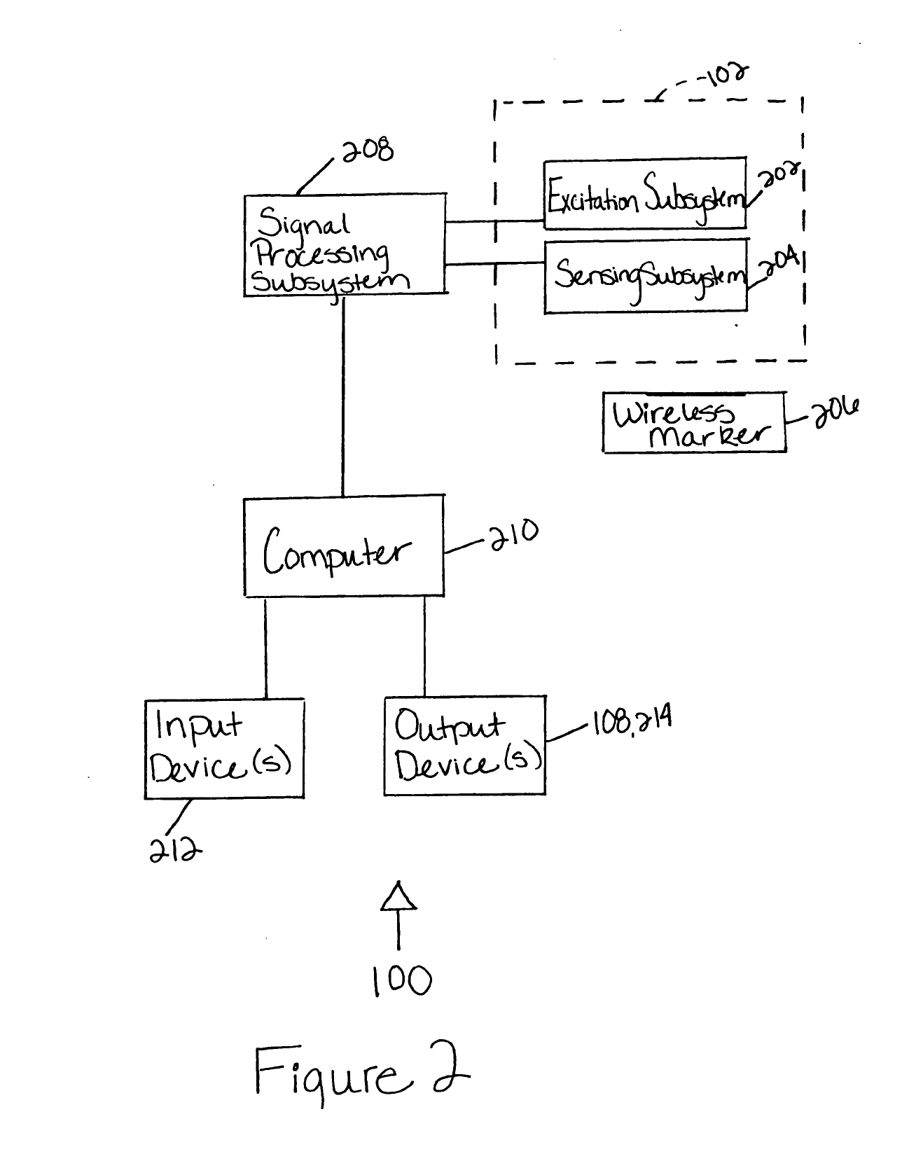

FIG. 2 is a block diagram of certain components of the system 100. In particular, the excitation source and sensor array 102 includes an excitation subsystem 202 and a sensing subsystem 204. The excitation system 202 outputs electromagnetic energy to excite at least one wireless marker 206, and the sensing system 204 receives electromagnetic energy from the marker. Details regarding the sensing subsystem 204 are provided below.

A signal processing subsystem 208 provides signals to the excitation subsystem 202 to generate the excitation signals. In the embodiment depicted herein, excitation signals in the range of 300 to 500 kilohertz may be used. The signal processing subsystem 208 also receives signals from the sensing subsystem 204. The signal processing subsystem 208 filters, amplifies and correlates the signals received from the sensing subsystem 204 for use in a computer 210.

The computer 210 may be any suitable computer, such as an industrial personal computer suitable for medical applications or environments. One or more input devices 212 are coupled to the computer and receive user input. Examples of such input devices 212 include keyboards, microphones, mice/track balls, joy sticks, etc. The computer generates output signals provided to output devices 214. Examples of such output devices include the display device 108, as well as speakers, printers, and network interfaces or subsystems to connect the computer with other systems or devices.

Unless described otherwise herein, several aspects of the invention may be practiced with conventional systems. Thus, the construction and operation of certain blocks shown in FIG. 2 may be of conventional design, and such blocks need not be described in further detail to make and use the invention because they will be understood by those skilled in the relevant art.

Description of Suitable Sensing Subsystems

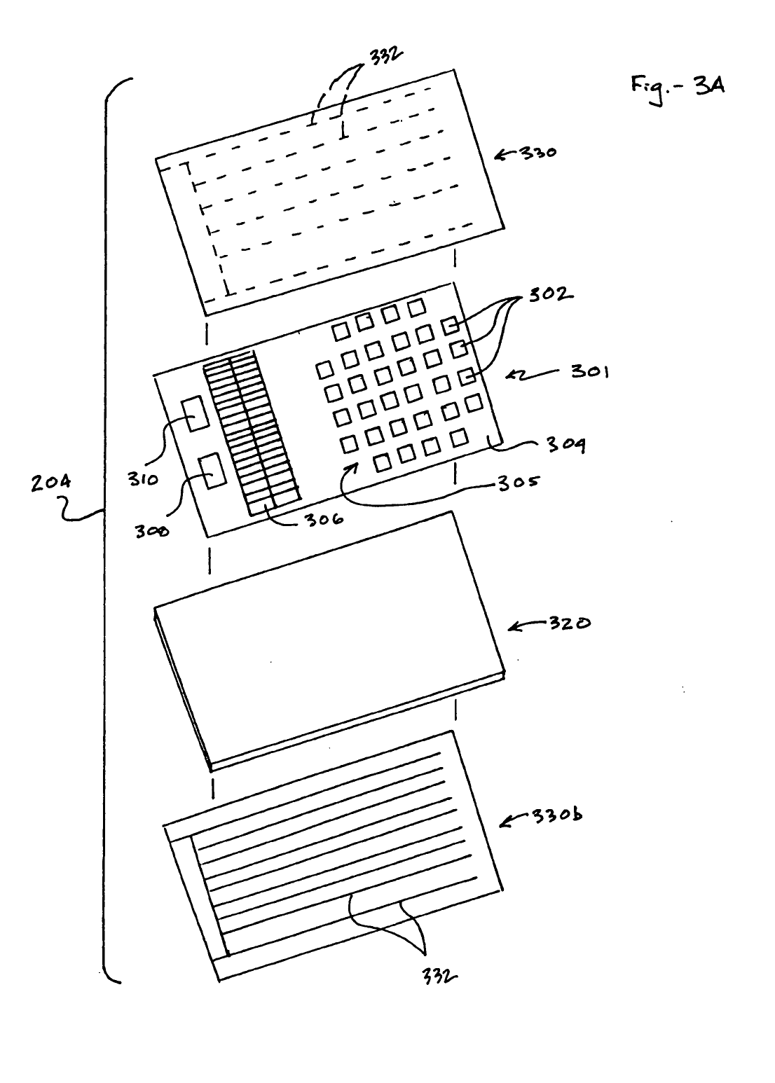

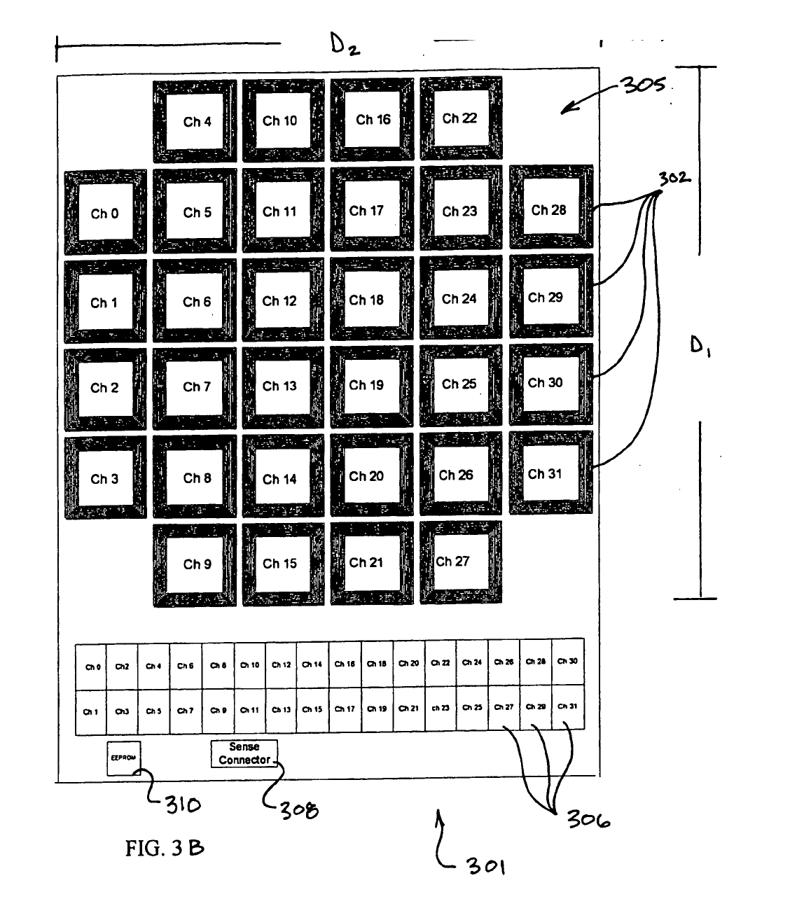

FIG. 3A is an exploded isometric view showing several components of the sensing subsystem 204. The subsystem 204 includes a sensing assembly 301 having a plurality of coils 302 formed on or carried by a panel 304. The coils are arranged in a sensor array 305. The panel 304 may be a substantially non-conductive sheet, such as KAPTON® produced by DuPont. KAPTON® is particularly useful when an extremely stable, tough, and thin film is required (such as to avoid radiation beam contamination), but the panel 304 may be made from other materials. For example, FR4 (epoxy-glass substrates), GETEK and Teflon-based substrates, and other commercially available materials can be used for the panel 304. Additionally, although the panel 304 may be a flat, highly planar structure, in other embodiments, the panel may be curved along at least one axis. In either embodiment, the panel is at least substantially locally planar such that the plane of one coil is at least substantially coplanar with the planes of adjacent coils. For example, the angle between the plane defined by one coil relative to the planes defined by adjacent coils can be from approximately 0° to 10°, and more generally is less than 5°. In some circumstances, however, one or more of the coils may be at an angle greater than 10° relative to other coils in the array.

The sensing subsystem 204 shown in FIG. 3A can further include a low-density foam spacer or core 320 laminated to the panel 304. The foam core 320 can be a closed-cell Rohacell foam. The foam core 320 is preferably a stable layer that has a low coefficient of thermal expansion so that the shape of the sensing subsystem 204 and the relative orientation between the coils 302 remains within a defined range over an operating temperature range.

The sensing subsystem 204 can further include a first exterior cover 330a on one side of the sensing subsystem and a second exterior cover 330b on an opposing side. The first and second exterior covers 330a-b can be thin, thermally stable layers, such as Kevlar or Thermount films. Each of the first and second exterior covers 330a-b can include electric shielding 332 to block undesirable external electric fields from reaching the coils 302. The electric shielding, for example, prevents or minimizes the presence of eddy currents caused by the coils 302 or external electric fields. The electric shielding can be a plurality of parallel legs of gold-plated, copper strips to define a comb-shaped shield in a configuration commonly called a Faraday shield. It will be appreciated that the shielding can be formed from other materials that are suitable for shielding. The electric shielding can be formed on the first and second exterior covers using printed circuit board manufacturing technology or other techniques.

The panel 304 with the coils 302 is laminated to the foam core 320 using an epoxy or another type of adhesive. The first and second exterior covers 330a-b are similarly laminated to the assembly of the panel 304 and the foam core 320. The laminated assembly forms a rigid, lightweight structure that fixedly retains the arrangement of the coils 302 in a defined configuration over a large operating temperature range. As such, the sensing subsystem 204 does not substantially deflect across its surface during operation. The sensing subsystem 204, for example, can retain the array of coils 302 in the fixed position with a deflection of no greater than ±0.5 mm, and in some cases no more than ±0.3 mm. The stiffness of the sensing subsystem 204 provides very accurate and repeatable monitoring of the precise location of leadless markers in real time.

The sensing subsystem 204 can also have a low mass per unit area in the plane of the sensor coils 302. The “mass-density” is defined by the mass in a square centimeter column through the thickness of the sensing subsystem 204 orthogonal to the panel 304. In several embodiments, the sensing subsystem 204 has a low-density in the region of the coils 302 to allow at least a portion of the sensing subsystem 204 to dwell in a radiation beam of a linear accelerator used for radiation oncology. For example, the portion of the sensing subsystem 204 including the coils 302 can have a mass density in the range of approximately 1.0 gram/cm² or less. In general, the portion of the sensing subsystem that is to reside in the beam of a linear accelerator has a mass-density between approximately 0.1 grams/cm² and 0.5 grams/cm², and often with an average mass-density of approximately 0.3 grams/cm². The sensing subsystem 204 can accordingly reside in a radiation beam of a linear accelerator without unduly attenuating or contaminating the beam. In one embodiment, the sensing subsystem 204 is configured to attenuate a radiation beam by approximately only 0.5% or less, and/or increase the skin dose in a patient by approximately 80%. In other embodiments, the panel assembly can increase the skin dose by approximately 50%. Several embodiments of the sensing subsystem 204 can accordingly dwell in a radiation beam of a linear accelerator without unduly affecting the patient or producing large artifacts in x-ray films.

In still another embodiment, the sensing subsystem 204 can further include a plurality of source coils that are a component of the excitation subsystem 202. One suitable array combining the sensing subsystem 204 with source coils is disclosed in U.S. patent application Ser. No. 10/334,700, entitled PANEL-TYPE SENSOR/SOURCE ARRAY ASSEMBLY, filed on Dec. 30, 2002, which is herein incorporated by reference.

FIG. 3B further illustrates an embodiment of the sensing assembly 301. In this embodiment, the sensing assembly 301 includes 32 sense coils 302; each coil 302 is associated with a separate channel 306 (shown individually as channels “Ch 0 through Ch 31”). The overall dimension of the panel 304 can be approximately 40 cm by 54 cm, but the array 305 has a first dimension D₁ of approximately 40 cm and a second dimension D₂ of approximately 40 cm. The coil array 305 can have other sizes or other configurations (e.g., circular) in alternative embodiments. Additionally, the coil array 305 can have more or fewer coils, such as 8-64 coils; the number of coils may moreover be a power of 2.

The coils 302 may be conductive traces or depositions of copper or another suitably conductive metal formed on the KAPTON® sheet. Each coil 302 has traces with a width of approximately 0.15 mm and a spacing between adjacent turns within each coil of approximately 0.15 mm. The coils 302 can have approximately 15 to 90 turns, and in specific applications each coil has approximately 40 turns. Coils with less than 15 turns may not be sensitive enough for some applications, and coils with more than 90 turns may lead to excessive voltage from the source signal during excitation and excessive settling times resulting from the coil's lower self-resonant frequency. In other applications, however, the coils 302 can have less than 15 turns or more than 90 turns.

As shown in FIG. 3B, the coils 302 are arranged as square spirals, although other configurations may be employed, such as arrays of circles, interlocking hexagons, triangles, etc. Such square spirals utilize a large percentage of the surface area to improve the signal to noise ratio. Square coils also simplify design layout and modeling of the array compared to circular coils; for example, circular coils could waste surface area for linking magnetic flux from the wireless markers 206. The coils 302 have an inner diameter of approximately 40 mm, and an outer diameter of approximately 62 mm, although other dimensions are possible depending upon applications. Sensitivity may be improved with an inner diameter as close to an outer diameter as possible given manufacturing tolerances. In several embodiments, the coils 32 are identical to each other or at least configured substantially similarly.

The pitch of the coils 302 in the coil array 305 is a function of, at least in part, the minimum distance between the marker and the coil array. In one embodiment, the coils are arranged at a pitch of approximately 67 mm. This specific arrangement is particularly suitable when the wireless markers 206 are positioned approximately 7-27 cm from the sensing subsystem 204. If the wireless markers are closer than 7 cm, then the sensing subsystem may include sense coils arranged at a smaller pitch. In general, a smaller pitch is desirable when wireless markers are to be sensed at a relatively short distance from the array of coils. The pitch of the coils 302, for example, is approximately 50%-200% of the minimum distance between the marker and the array.

In general, the size and configuration of the coil array 305 and the coils 302 in the array 305 depend on the frequency range in which they are to operate, the distance from the wireless markers 206 to the array, the signal strength of the markers, and several other factors. Those skilled in the relevant art will readily recognize that other dimensions and configurations may be employed depending, at least in part, on a desired frequency range and distance from the markers to the coils.

The coil array 305 is sized to provide a large aperture to measure the magnetic field emitted by the markers. It can be particularly challenging to accurately measure the signal emitted by an implantable marker that wirelessly transmits a marker signal in response to a wirelessly transmitted energy source because the marker signal is much smaller than the source signal and other magnetic fields in a room (e.g., magnetic fields from CRTs, etc.). The size of the coil array 305 can be selected to preferentially measure the near field of the marker while mitigating interference from far field sources. In one embodiment, the coil array 305 is sized to have a maximum dimension D₁ or D₂ across the surface of the area occupied by the coils that is approximately 100% to 300% of a predetermined maximum sensing distance that the markers are to be spaced from the plane of the coils. Thus, the size of the coil array 305 is determined by identifying the distance that the marker is to be spaced apart from the array to accurately measure the marker signal, and then arrange the coils so that the maximum dimension of the array is approximately 100%-300% of that distance. The maximum dimension of the coil array 305, for example, can be approximately 200% of the sensing distance at which a marker is to be placed from the array 305. In one specific embodiment, the marker 206 has a sensing distance of 20 cm and the maximum dimension of the array of coils 302 is between 20 cm and 60 cm, and more specifically 40 cm.

A coil array with a maximum dimension as set forth above is particularly useful because it inherently provides a filter that mitigates interference from far field sources. It will be appreciated that in such a configuration the signal strength from the wireless marker decreases proportionally to the square of the distance. However, far field signals from electromagnetic noise generated by other systems in the environment decrease proportionally to the cube of the distance. Thus, when the wireless marker 206 is positioned approximately 20 cm from the sensing subsystem 204, and a radius or maximum dimension of the sensing subsystem is approximately 40 cm, signals from the wireless marker drop off at a square of the distance from the sensing subsystem while environmental noise drops off at a cube of the distance. The environmental noise is thus filtered by the sensing subassembly 204 to provide better signals to the signal processing subsystem 208.

The size or extent of the array may be limited by several factors. For example, the size of the sensing assembly 301 should not be so large as to mechanically interfere with the movable arm 104 (FIG. 1), the base unit 106 (FIG. 1), or other components, such as a patient couch, rotating gantry of a radiation therapy machine, etc. (not shown in FIG. 1). Also, the size of the array may be limited by manufacturing considerations, such as a size of available panels 304. Further, making a dimension or width of the coil array 305 larger than twice the distance to the wireless marker 206 may yield little performance improvement, but increase manufacturing costs and increase sensitivity to interference.

The coils 302 are electromagnetic field sensors that receive magnetic flux produced by the wireless marker 206 and in turn produce a current signal representing or proportional to an amount or magnitude of a component of the magnetic field through an inner portion or area of each coil. The field component is also perpendicular to the plane of each coil 302. Importantly, each coil represents a separate channel, and thus each coil outputs signals to one of 32 output ports 306. A preamplifier, described below, may be provided at each output port 306. Placing preamplifiers (or impedance buffers) close to the coils minimizes capacitive loading on the coils, as described herein. Although not shown, the sensing assembly 301 also includes conductive traces or conductive paths routing signals from each coil 302 to its corresponding output port 306 to thereby define a separate channel. The ports in turn are coupled to a connector 308 formed on the panel 304 to which an appropriately configured plug and associated cable may be attached.

The sensing assembly 301 may also include an onboard memory or other circuitry, such as shown by electrically erasable programmable read-only memory (EEPROM) 310. The EEPROM 310 may store manufacturing information such as a serial number, revision number, date of manufacture, and the like. The EEPROM 310 may also store per-channel calibration data, as well as a record of run-time. The run-time will give an indication of the total radiation dose to which the array has been exposed, which can alert the system when a replacement sensing subsystem is required.

While shown in only one plane, additional coils or electromagnetic field sensors may be arranged perpendicular to the panel 304 to help determine a three-dimensional location of the wireless markers 206. Adding coils or sensors in other dimensions could increase total energy received from the wireless markers 206 by 3 dB. However, the complexity of such an array may increase three-fold. The inventors have found that three-dimensional coordinates of the wireless markers 206 may be found using the planar array shown in FIG. 3B.

Amplification of Signals from Coils

Implementing the sensing subsystem 204 may involve several considerations. First, the coils 302 may not be presented with an ideal open circuit. Instead, they may well be loaded by parasitic capacitance due largely to traces or conductive paths connecting the coils to the preamplifiers, as well as a damping network (described below) and an input impedance of the preamplifiers (although a low input impedance is preferred). These combined loads result in current flow when the coils 302 link with a changing magnetic flux. Any one sense coil 302, then, links magnetic flux not only from the wireless marker 206, but also from all the other sense coils as well. These current flows should be accounted for in downstream signal processing. Thus, the calibration method and apparatus of the present invention addresses this issue, as well as others.

A second consideration is the capacitive loading on the coils 302. In general, it is desirable to minimize the capacitive loading on the coils 302. Capacitive loading forms a resonant circuit with the coils themselves, which leads to excessive voltage overshoot when the excitation subsystem 202 is energized. Such a voltage overshoot should be limited or attenuated with a damping or “snubbing” network across the coils 302. A greater capacitive loading requires a lower impedance damping network, which can result in substantial power dissipation and heating in the damping network.

Another consideration is to employ preamplifiers that are low noise. The preamplification may also be radiation tolerant because one application for the sensing subsystem 204 is with radiation therapy systems that use linear accelerators (LINAC). As a result, PNP bipolar transistors and discrete elements may be preferred. Further, a DC coupled circuit may be preferred if good settling times cannot be achieved with an AC circuit or output, particularly if analog to digital converters are unable to handle wide swings in an AC output signal.

Calibration of Sensing Array

Calibration Architecture

FIG. 4 is a schematic of a single differential preamplifier 404 and associated calibration sources for use with a sensing element or sensing coil of the sensing array. Also included is a snubbing network 402 that includes two pairs of series coupled resistors and a capacitor bridging therebetween. As will be seen below, calibration inputs 408 and 412 are used to provide voltage and current calibration signals, respectively. The sensor coil 302 is coupled to an input of the differential amplifier 404, followed by a pair of high voltage protection diodes 410. DC offset may be adjusted by a pair of resistors coupled to bases of the input transistors for the differential amplifier 404 (R_{div1}).

In one embodiment, the precision calibration sources are implemented by switching square-wave waveforms into any of four injection ports per differential preamplifier channel. In one embodiment, the square-wave waveforms are filtered to produce a sinusoidal waveform. The two injection ports through large-valued, precision resistors Rref make up the I_{CAL}, or current injection calibration ports 408. The two injection ports through precision resistor dividers (R_{DIV1} and R_{DIV2}) constitue the V_{CAL}, or voltage calibration ports 412. The square-wave waveforms are generated with registers implemented in the AC family of CMOS logic. This family of logic switches the register outputs to either GND or V_{S} through very low-valued switch resistances. A stable and accurate supply voltage V_{S} guarantees that square-waves of precise amplitude and phase are generated. The calibration sources can be activated in any desired pattern, either singly, differentially, or in common-mode. As noted above, the receiver 208 contains filtering to eliminate all higher harmonics leaving only the fundamental of the calibration waveform. All signal-path circuitry is operated in a highly linear manner. Additionally, while a square wave form is used as the injected signal, other waveforms may be suitable for the calibration process.

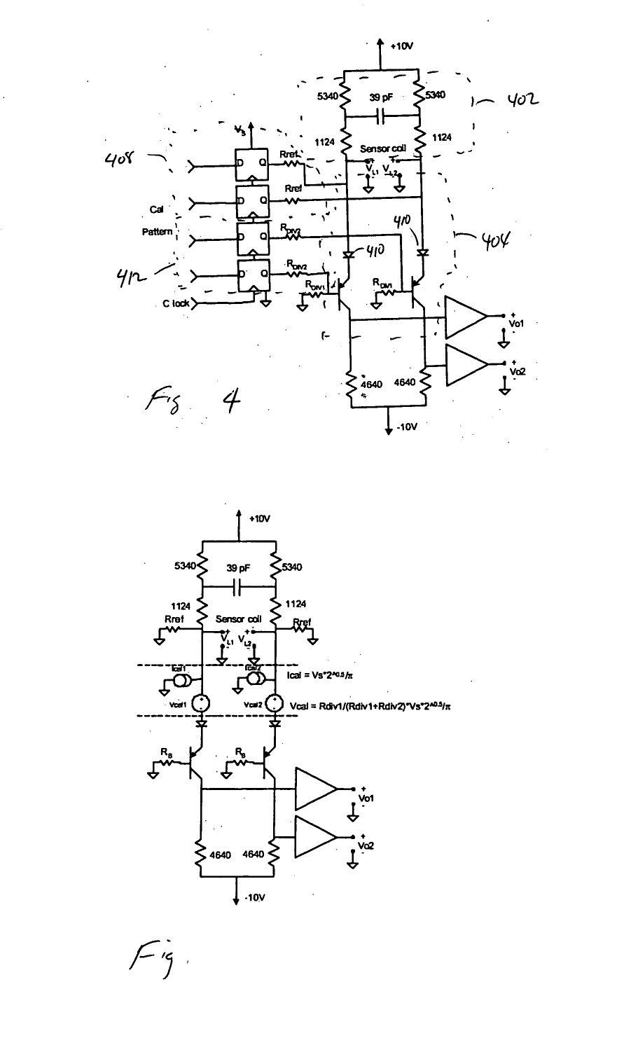

FIG. 5 is an equivalent circuit of the single differential preamplifier of FIG. 4, which can be derived using the hyprid-pi model assuming no impedance between the collector and base terminals of the transistor. The square-wave calibration sources have been replaced by idealized sinusoidal voltage and current sources with root mean square (RMS) amplitudes related to the supply voltage V_{S}. As will be seen below, by selectively injecting voltage or current signals (referred to also as an excitation signal) across one sensing coil and into the amplifier, the effect on all of the sense coils 302 can be ascertained by receiver 208. Once this is done, then a correction (calibration) matrix can be generated.

In an alternative embodiment, more than one sensing coil may be injected with voltage or current excitation signal simultaneously and the effect on the sense coils 302 may be measured. In yet another alternative embodiment, only the effect on some of the sense coils 302 is monitored and analyzed. Thus, while the specific embodiment described herein discusses injection of an excitation signal into one sense coil and monitoring the effect on all of the sense coils 302, it is contemplated that various combinations of excitation and monitoring may be implemented while still staying within the spirit of the present invention.

Further, while in the embodiment described herein teaches that each of the sense coils 302 is injected with an excitation, in an alternative embodiment, less than all of the sense coils 302 of the sensing array are excited. For example, in one embodiment, only 16 of the 32 sense coils 302 are excited. The 16 excited sense coils form a calibration subset of the sense coils. Indeed, the term calibration subset may include all of the sense coils 302 in the sensing array, and thus, the term calibration subset may mean anywhere from one or all of the sense coils in the sensing array.

Note that in the description herein, the calculations may be performed in the signal processing subsystem 208 or in some other processing device. In one embodiment, the calculations are implemented by a digital signal processor. However, in alternative embodiments, the processing or analysis can be done using programmable logic devices or even software running on a general purpose microprocessor.

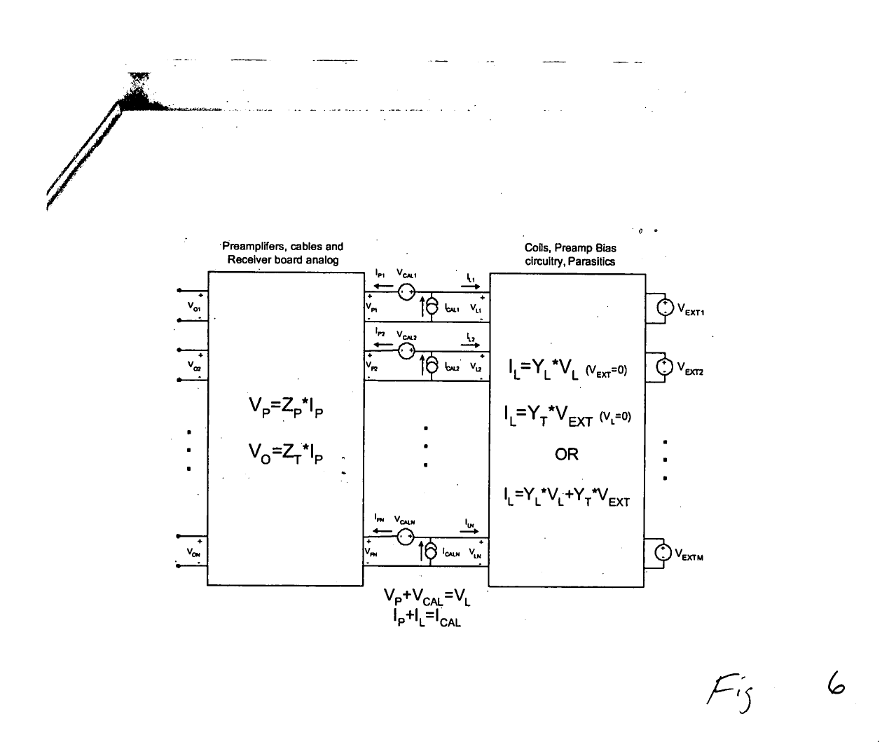

FIG. 6, depicts the entire M=32 coil array, N=64 current calibration sources, N=64 voltage calibration sources and the network consisting of N/2=32 differential preamplifiers. As indicated by the dashed lines in FIG. 5, for analysis purposes, the Rref resistor, snubber and biasing network are incorporated in the Y_{L} array matrix. The V_{EXT} vector represents the induced open-circuit Faraday voltage in the M sense coils. The N×M Y_{T} ‘transadmittance’ matrix relates the N short-circuit currents I_{L} measured at the calibration current injection points to the M elements of V_{EXT}. N×N Y_{L} represents the ‘self-admittance’ of the array as measured from the calibration current injection points. Note that the N=2*M 2-ports on the left of the array can be considered as single-ended signals referred to a common reference, or as M differential ports and M common-mode ports referred to a common reference (with consistent representations of Y_{L} and Y_{T}).

Similarly, the preamplifiers, cables and receiver board are characterized by an N×N (64×64) complex ‘transimpedance’ Z_{T} matrix, an N×N complex ‘self-impedance’ Z_{P} matrix, N×1 output voltage vector V_{O}, N×1 input voltage vector V_{P} and N×1 input current I_{P}.

The N calibration current sources I_{CAL} and N calibration voltage sources V_{CAL} are related to the array and preamp voltages and currents by the nodal equations shown in FIG. 6.

During normal (marker localization) operation, the calibration sources 408 and 412 (V_{CAL}=0 and I_{CAL}=0) are set to zero. Further, the output voltage vector V_{O} and the ‘hypothetical’ short-circuit current I_{L}_{_{—}}_{SC} can be found using the equations in FIG. 6 from the V_{EXT} voltages:

$$\begin{matrix}{V}_{O} = - {Z}_{T} \cdot {( {I}_{M \times M} + {Y}_{L} \cdot {Z}_{P} )}^{- 1} \cdot {Y}_{T} \cdot {V}_{EXT} \\ = - {T}^{- 1} \cdot {Y}_{T} \cdot {V}_{EXT} \\ {I}_{L\_SC} = {Y}_{T} \cdot {V}_{EXT} \\ = - T \cdot {V}_{O}\end{matrix}$$

Thus, multiplying the measured output voltage vector by T gives the short-circuit current measurement of the array. T⁻¹ is defined as follows and is measured during calibration:
T⁻¹=Z_{T}·(I_{M×M}+Y_{L}·Z_{P})⁻¹

During excitation of the calibration currents, with V_{CAL}=0 and V_{EXT}=0:

$$\begin{matrix}{V}_{O} = {Z}_{T} \cdot {( {I}_{M \times M} + {Y}_{L} \cdot {Z}_{P} )}^{- 1} \cdot {I}_{CAL} \\ = {T}^{- 1} \cdot {I}_{CAL}\end{matrix}$$

During excitation of the calibration voltage, with I_{CAL}=0 and V_{EXT}=0:

$$\begin{matrix}{V}_{O} = - {Z}_{T} \cdot {( {I}_{M \times M} + {Y}_{L} \cdot {Z}_{P} )}^{- 1} \cdot {Y}_{L} \cdot {V}_{CAL} \\ = - {T}^{- 1} \cdot {Y}_{L} \cdot {V}_{CAL}\end{matrix}$$

A calibration sequence consisting of measuring column vector V_{O} while stepping through each of the I_{CAL} elements one at a time measures the columns of T⁻¹. Stepping through each of the V_{CAL} elements one at a time measures the columns of T⁻¹*Y_{L}. This architecture then allows the measurement of Y_{L}, and the measurement of the short-circuit current resulting from V_{EXT}: I_{L}_{_{—}}_{SC}=Y_{T}*V_{EXT}. Thus, if a relation can be derived for the measured N×N Y_{L} to the needed N×M Y_{T}, then it can be determined, with high accuracy, the original V_{EXT}.

Single-Ended Representation vs. Differential-Mode/Common-Mode Representation:

In one embodiment, fully differential circuit topologies is used throughout the array and receive signal chain. As seen above, the preamplifier is a differential amplifier with opposing bipolar transistors. Thus, there are advantages to representing the Z, Y and T matrices noted above with M=32 differential and M=32 common mode ports, rather than N=2M=64 single-ended ports. Each port always consists of two terminals. The single-ended and common-mode ports always use the common ground reference as one terminal, the differential ports do not use a ground reference.

Denoting I₊, I₋, V₊ and V₋ as 32×1 column vectors corresponding to the positive and negative single-ended terminal currents and voltages for the 32 pairs of ports of the Y matrix, the 64×1 column vectors and 64×64 Y matrix segmented can be created as follows:

$$[ \frac{{I}_{+}}{{I}_{-}} ] = [ \frac{{Y}_{++} \vert {Y}_{+ -}}{{Y}_{- +} \vert {Y}_{--}} ] [ \frac{{V}_{+}}{{V}_{-}} ]$$

Alternately, the coordinate transformation to differential and common-mode currents and voltages may be performed as:

$${I}_{D} = ( {I}_{+} - {I}_{-} ) / 2$$

$${I}_{C} = ( {I}_{+} + {I}_{-} ) / 2$$

$${V}_{D} = {V}_{+} - {V}_{-}$$

$${V}_{C} = {V}_{+} + {V}_{-}$$

$$\begin{matrix}[ \frac{{I}_{D}}{{I}_{C}} ] = [ \frac{{Y}_{DD} \vert {Y}_{D C}}{{Y}_{CD} \vert {Y}_{CC}} ] [ \frac{{V}_{D}}{{V}_{C}} ] \\ = \frac{1}{4} [ \frac{{Y}_{++} + {Y}_{--} - {Y}_{+ -} - {Y}_{- +} \vert {Y}_{++} - {Y}_{--} + {Y}_{+ -} - {Y}_{- +}}{{Y}_{++} - {Y}_{--} + {Y}_{+ -} - {Y}_{- +} \vert {Y}_{++} + {Y}_{--} + {Y}_{+ -} + {Y}_{- +}} ] [ \frac{{V}_{D}}{{V}_{C}} ]\end{matrix}$$

Y_{DD} represents the short-circuit differential current for a differential voltage drive.

Y_{CC} represets the short-circuit common-mode current for a common-mode voltage drive.

Y_{DC} and Y_{CD} represent the mode coupling admittances.

Model of Array Admittances:

This section will discuss the relation of the measurable Y_{L} (admittance measured from the array coil terminals) to the desired Y_{T} (the transadmittance from the Faraday induced voltage to the array coil terminals).

A number of different intentional circuit elements and unintentional (parasitic) circuit elements contribute to the total Y_{L}. It can be shown that the admittances from these other elements simply add to the desired transadmittance, Y_{T}. Determining Y_{T} from the measured Y_{L} just involves determining and subtracting off the undesired components of Y_{L}.

Three types of array admittances can be identified with respect to calculating the desired Y_{T}.
Y_{L}=Y_{L1}+Y_{L2}+Y_{L3}

(1) The ‘ideal’ sense coils themselves: These components have no common mode admittance and no common to differential or differential to common mode coupling terms. Note that the 64×32 Y_{T} is just the first 32 columns of Y_{L1}.

$${Y}_{L 1} = [ \frac{{Y}_{L 1 DD} \vert {Y}_{L 1 D C}}{{Y}_{L 1 CD} \vert {Y}_{L 1 CC}} ] = [ \begin{matrix}{M}_{C}^{- 1} & \vert & 0 \\ 0 & \vert & 0\end{matrix} ]$$

$${Y}_{T} = [ \frac{{Y}_{TDD}}{{Y}_{TCD}} ] = [ \frac{{M}_{C}^{- 1}}{0} ]$$

(2) Circuit elements which are connected directly between array coil terminals and the common reference ground potential: These components have a differential-mode admittance that is equal to the common-mode admittance. The differential mode components can be determined by measurement of the common-mode components of Y_{L}. Examples in this category include:
Preamp bias resistors
Impedance of the Norton equivalent resistor for the current calibration source Rref
Stray capacitance to GND of differential pairs and coil

All these components show up only on the diagonal of each block. In general, these components are reasonably well matched (˜1%) and fairly small to start with. If the shunt impedances to GND are denoted z₁ₚ, z₁ₙ, z₂ₚ, z₂ₙ, . . . z_{Np}, z_{Nn}, then the admittance matrix is:

$${Y}_{L 2} = [ \frac{{Y}_{DD} \vert {Y}_{D C}}{{Y}_{CD} \vert {Y}_{CC}} ] = \frac{1}{4} [ \begin{matrix}{z}_{1 p}^{- 1} + {z}_{1 n}^{- 1} & 0 & ⋯ & 0 & \vert & {z}_{1 p}^{- 1} - {z}_{1 n}^{- 1} & 0 & ⋯ & 0 \\ 0 & {z}_{2 p}^{- 1} + {z}_{2 n}^{- 1} & ⋯ & 0 & \vert & 0 & {z}_{2 p}^{- 1} - {z}_{2 n}^{- 1} & ⋯ & 0 \\ ⋮ & ⋮ & ⋰ & ⋮ & \vert & ⋮ & ⋮ & ⋰ & ⋮ \\ 0 & 0 & ⋯ & {z}_{N p}^{- 1} + {z}_{Nn}^{- 1} & \vert & 0 & 0 & ⋯ & {z}_{N p}^{- 1} - {z}_{Nn}^{- 1} \\ {z}_{1 p}^{- 1} - {z}_{1 n}^{- 1} & 0 & ⋯ & 0 & \vert & {z}_{1 p}^{- 1} + {z}_{1 n}^{- 1} & 0 & ⋯ & 0 \\ 0 & {z}_{2 p}^{- 1} - {z}_{2 n}^{- 1} & ⋯ & 0 & \vert & 0 & {z}_{2 p}^{- 1} + {z}_{2 n}^{- 1} & ⋯ & 0 \\ ⋮ & ⋮ & ⋰ & ⋮ & \vert & ⋮ & ⋮ & ⋰ & ⋮ \\ 0 & 0 & ⋯ & {z}_{N p}^{- 1} - {z}_{Nn}^{- 1} & \vert & 0 & 0 & ⋯ & {z}_{N p}^{- 1} + {z}_{Nn}^{- 1}\end{matrix} ]$$

(3) Those elements whose common-mode and differential-mode admittances are not the same, and hence must be characterized during engineering or manufacturing and included in an EEPROM with the system 100. There are two types, which will be modeled separately.
Y_{L3}=Y_{L3A}+Y_{L3B}

The first type, Y_{L3A} covers the differential shunt impedance across each coil. This is from the intentional snubber network, and the unintentional parasitic capacitance across the coil and differential pairs leading from the coil. If these individual impedances are denoted z₃₁, z₃₂, . . . z_{3N}, the admittance matrix is:

$${Y}_{L 3 A} = [ \frac{{Y}_{DD} \vert {Y}_{D C}}{{Y}_{CD} \vert {Y}_{CC}} ] = [ \begin{matrix}{z}_{31}^{- 1} & 0 & ⋯ & 0 & \vert & 0 & 0 & ⋯ & 0 \\ 0 & {z}_{32}^{- 1} & ⋯ & 0 & \vert & 0 & 0 & ⋯ & 0 \\ ⋮ & ⋮ & ⋰ & ⋮ & \vert & ⋮ & ⋮ & ⋰ & ⋮ \\ 0 & 0 & ⋯ & {z}_{3 N}^{- 1} & \vert & 0 & 0 & ⋯ & 0 \\ 0 & 0 & ⋯ & 0 & \vert & 0 & 0 & ⋯ & 0 \\ 0 & 0 & ⋯ & 0 & \vert & 0 & 0 & ⋯ & 0 \\ ⋮ & ⋮ & ⋰ & ⋮ & \vert & ⋮ & ⋮ & ⋰ & ⋮ \\ 0 & 0 & ⋯ & 0 & \vert & 0 & 0 & ⋯ & 0\end{matrix} ]$$

The second type, Y_{L3B}, covers the small capacitive coupling between the differential pairs and coils in the array. Unfortunately, this is not expected to appear mainly common-mode or differential mode. However it will have 0's on the diagonals of each block. This type is characterized in differential mode and stored in EEPROM.

Thus to determine the desired Y_{TDD}=M_{C}⁻¹, the measured differential Y_{LDD} is taken and the Y_{L3ADD} and Y_{L3BDD} derived from the EEPROM is subtracted. Further, also subtracted are the diagonal elements of the measured common-mode Y_{LCCdiagonal},
Y_{TDD}=M⁻¹_{c}=Y_{L}_{_{DD}}−Y_{L3B}_{_{DD}}−Y_{L}_{_{CC}}_{—diagonal}

Note that only the diagonal elements of the measured Y_{LCC} should be subtracted off. The off-diagonal elements include common-mode capacitance (Y_{L3BCC}) between the coils that do not affect Y_{T}.

Dynamic vs. Static Calibration:

Some of the array and receiver parameters discussed above are subject to variation with temperature (such as the preamplifier gain and input impedance represented by Z_{T}, Z_{P} and T). Other parameters such as Y_{T}, Y_{L} or M_{C} are subject to variation on much shorter time scales as metallic objects may be introduced to or removed from the vicinity of the array. Still others are expected to remain essentially static over time, such as Y_{LCC}. It is therefore important to execute measurements of the time-varying parameters interleaved with marker localization measurements to assure accurate calibration of the system 100. In other words, the calibration of the array is done in real time as the array is being used for marker localization.

Executing calibration measurements frequently, however, reduces the percentage of time during which localization measurements can be made, which in turn will impact adversely either the update rate or variance of the localization measurements.

As only one block of the Y_{L} matrix, Y_{LDD}, is prone to variation, considerable time can be saved by exciting the VCAL sources in differential mode only, and making only the differential receive measurement. This reduces by approximately 75% the time required to make the voltage calibration measurements.

A similar 75% savings can be achieved in the current calibration measurements if the T matrix is block-diagonal. In the calibration process outlined above, the columns of T⁻¹ are measured sequentially, and the desired T matrix is calculated with a matrix inversion. In general, the full T⁻¹ matrix must be measured to determine T, but if T⁻¹ is block-diagonal, the diagonal blocks can be measured and inverted independently.

$${[ \begin{matrix}{Tinv}_{DD} & 0 \\ 0 & {Tinv}_{CC}\end{matrix} ]}^{- 1} = [ \begin{matrix}{Tinv}_{DD}^{- 1} & 0 \\ 0 & {Tinv}_{CC}^{- 1}\end{matrix} ]$$

Experimental measurements of the T⁻¹ matrix indicate that the off-diagonal blocks are 40 to 50 dB smaller than the diagonal blocks. The error introduced in making the block-diagonal assumption in the matrix inversion goes as the square of the relative magnitudes of the off-diagonal blocks. Errors of less than 100 ppm can be expected in making this assumption.

Thus only differential-mode excitation and differential-mode receive measurements need to be made dynamically. The full calibration need only be executed on a much less frequent basis, such as once per patient, once per day, or even just once in the factory.

Mitigation of Channel-To-Channel Mismatch in Voltage Calibration Sources:

In characterizing Y_{L} one column at a time, the ‘short-circuit’ current is measured while driving one channel (differentially or common-mode) at a time. Mismatches in the individual (single-ended) calibration voltage sources result in the following effects:
Channel-channel amplitude mismatch in the differential drive voltages
Channel-channel amplitude mismatch in the common-mode drive voltages.
Crosstalk into the common mode when driving differentially
Crosstalk into the differential mode when driving common-mode.

Crosstalk between modes is treated in the next section.

Mismatches in the current calibration sources are expected to be limited by the accuracy of the R_{REF} resistor, or about 0.02%. Achieving this type of accuracy in the VCAL voltage dividers, though, is not feasible.

Mismatches in the voltage divider unique to each channel will result in gain errors common to each column of the measured admittance. Let Y_{M} be the measured (corrupted) admittance, and G_{V}⁻¹ be the (complex) diagonal matrix representing the (unknown) gain errors that caused the corruption:
Y_{M}=Y_{L}·G_{V}⁻¹
Y_{L}=Y_{M}·G_{V}

We want to find that G_{V}=diag{[g₁ g₂ g₃ . . . g₃₂]} to correct the measured Y_{M} and achieve the true Y_{L}. Reciprocity will guarantee that Y_{L} is a symmetric matrix. A least squares fit can be applied to ‘back out’ the differential voltage drive mismatches that were present. The equations describing reciprocity of Y_{L} are:

$$\begin{matrix}{Y}_{L 12} = {Y}_{L 21} \\ {Y}_{L 13} = {Y}_{L 31} \\ ⋮\end{matrix} or \begin{matrix}{Y}_{M 12} {g}_{2} = {Y}_{M 21} {g}_{1} \\ {Y}_{M 13} {g}_{3} = {Y}_{M 31} {g}_{1} \\ ⋮\end{matrix}$$

This is a system of 1/2*(32\hat{ }2−32)=496 equations with only 32 (complex) unknowns (g₁ . . . g₃₂). Of course the least squares fit to this system of equations will be g₁=g₂= . . . g₃₂=0. To assure the solution we want, we need to add one more equation that assures the mean of the gains is 1: g₁+g₂+ . . . g₃₂=32.

This system of equations can be captured in matrix form as:
A·g=b
with:

$$g = {[ \begin{matrix}{g}_{1} & {g}_{2} & {g}_{3} & … & {g}_{31} & {g}_{32}\end{matrix} ]}^{T}$$

$$b = {[ \begin{matrix}0 & 0 & 0 & 0 & 0 & … & 0 & 0\end{matrix} \vert 32 ]}^{T}$$

$$A = [ \begin{matrix}{y}_{M21} & - {y}_{M12} & 0 & … & 0 & 0 \\ {y}_{M31} & 0 & - {y}_{M13} & … & 0 & 0 \\ 0 & {y}_{M32} & - {y}_{M23} & … & 0 & 0 \\ ⋮ & ⋮ & ⋮ & ⋮ & ⋮ & ⋮ \\ 0 & 0 & 0 & … & {y}_{M3231} & - {y}_{M3132} \\ 1 & 1 & 1 & … & 1 & 1\end{matrix} ]$$

The least squares solution to this overdetermined system of equations is:
g=(A^{H}A)⁻¹A^{H}b
G_{V}=diag{g}

Any differential-mode channel-channel mismatch should also be present in the common-mode measurement. The same voltage source error fit obtained to achieve a symmetrical Y_{L} in the differential block should be applied to the common-mode block as well. G_{V} may either be calculated as noted above, or in an alternative embodiment, may be calculated using other methodologies, such as by measuring the flatness of the sensing array and compensating therefore.

Effects of Differential Imbalance in the Calibration Drive Voltage Sources V_{CAL}:

Crosstalk from the differential mode to the common-mode can be analyzed as follows. When driving channel 1 differentially:

$${I}_{L\_SC} = - T \cdot {V}_{0} = {Y}_{L} \cdot {V}_{CAL} = [ \frac{{Y}_{DD} \vert {Y}_{DC}}{{Y}_{CD} \vert {Y}_{CC}} ] \cdot [ \begin{matrix}{V}_{CAL\_D} \\ {V}_{CAL\_C}\end{matrix} ]$$

$${V}_{CAL} = {[ \begin{matrix}V & 0 & … & 0\end{matrix} \vert ɛ V \begin{matrix}0 & … & 0\end{matrix} ]}^{T}$$

instead of measuring just the first column of Y_{DD}, we will also get □ times the first column of Y_{DC} added to it.

Y_{DC} contains only the mismatch in Y_{L2} shunt components, and Y_{L3B} stray capacitances. A rough order of magnitude estimate is given as follows: the shunt elements in Y_{L2} have 1/10 the admittance of the diagonal elements of Y_{DD}. The mismatch in these elements (which is what Y_{DC} contains) is probably <1/30 times that. Finally, ε is on the order of 1/1000. This makes this error term on the order of 3 ppm of the diagonal elements of Y_{DD}, and should be negligible.

Crosstalk from common-mode to differential mode will be the same order of magnitude.

Effect of Feedthrough and Crosstalk in the Calibration Drive Circuitry:

There are 4 types of feedthrough from calibration drive circuitry to the V_{O} output to consider:
Parasitic paths coupling the current-drive waveform into the V_{O} output—diagonal terms.
   The dominant path expected here is the small amount of capacitance across the R_{REF} resistor. Measurements indicate this is roughly 70 femtofarads (fF). R_{REF} is henceforth modeled as the actual resistance used (49.9 k in one embodiment) in parallel with 70 fF. At 500 kHz, this results in a gain error below 1 part per 10,000. Also, the layout of each channel is generally identical, so this gain error is assumed to be the same across all channels. This effect will be ignored (but verified in the hardware).
Parasitic paths coupling the current-drive waveform into the V_{O} output—off-diagonal terms.
   It is believed that this effect is negligible.
Parasitic paths coupling the voltage-drive waveform into the V_{O} output—diagonal terms.
   A significant effect is expected from the collector-base capacitance in the preamp PNP transistor. Errors from this source are mitigated by measuring and subtracting off the common-mode array admittance. In measuring the common-mode array admittance, the collector-base capacitance will appear as a ‘negative’ capacitance to GND in the array. This effect is present to the same degree in both differential and common-mode drive, so subtracting Y_{LCCDiagonal} from the measured Y_{LDD} is effective in compensating for this.
Parasitic paths coupling the voltage-drive waveform into the V_{O} output—off-diagonal terms.

There is also the potential for crosstalk between current-drive and voltage drive sources or between channels. However, it is believed that these effects are negligible.

Summary of EEPROM Stored Data:

In one actual embodiment, the following parameters have been developed for storage into non-volatile memory of the system 100. However, it can be appreciated that other sets of parameters would normally be developed for arrays that use differing design choices.

EEPROM Stored Data:
Rref: Current calibration reference impedance
Single real 16 bit value (in ohms)
   Nominal Value=49900 (49.9 Kohms)
Kdiv: Differential Voltage calibration divisor ratio.
   Single real 16 bit value (x1e-6)
   Nominal Value=6350 (0.006350)
R₁, R₃: Sense Coil snubber/bias resistance values.
   Two real 16 bit values (in ohms)
   Nominal Values=1124,5340
Csnub: Sense Coil snubber resistance value
   Single real 16 bit value (in 0.01 pF)
   Nominal Value=3900 (39 pF)
C_{3A}: Parasitic differential shunt capacitance across sense coils
   32 real 16 bit values (in 0.01 pF)
   Nominal Value=500 (5 pF)
C_{3B}: Differential mode parasitic capacitance between sense coils
   ˜100 non-zero values of upper-triangular 32×32 real matrix of 16 bit values (in 0.01 pF) (nearest neighbors only)
   Nominal Value=500 (5 pF) for nearest neighbors
Y_{LDD diagronal}: Results of dynamic cal in the factory at 300 kHz, 400 kHz, 500 kHz
   32×3 complex 16 bit values (in units of 100 nS)
   Nominal value at 300 kHz=6835−j*47240 (6.835e-4−j*4.724e-3 S)
   Nominal value at 400 kHz=3879−j*35751 (3.879e-4−j*3.575e-3 S)
   Nominal value at 500 kHz=2493−j*28721 (2.493e-4−j*2.872e-3 S)
Y_{LCC diagronal}: Results of static cal in the factory at 300 kHz, 400 kHz, 500 kHz
   32×3 complex 16 bit values (in units of 100 nS)
   Nominal value at 300 kHz=874−j*377 (8.74e-5−j*3.77e-5 S)
   Nominal value at 400 kHz=874−j*503 (8.74e-5−j*5.03e-5 S)
   Nominal value at 500 kHz=874−j*628 (8.74e-5−j*6.28e-5 S)

Calculation of Y_{L3A} and Y_{L3B} from EEPROM Stored Data:

Let s=j*2*pi*Fc where Fc is the operating center frequency (unique for each marker). For Y_{L3A}, the differential admittance of the snubber/bias network is calculated, and then the common mode admittance of the snubber/bias network (which is accounted for in the common-mode measurement) is subtracted off. This is a single parameter for all channels.

$${Y}_{SNUB} = \frac{1}{2 ( {R}_{1} + \frac{{R}_{3}}{2 {R}_{3} {C}_{SNUB} s + 1} )} - \frac{1}{2 ( {R}_{1} + {R}_{3} )}$$

To get Y_{L3A}, the per channel capacitances C_{3A} are added.
Y_{L3A}=Y_{SNUB}·I_{32×32}+diag{sC_{3A}}

Y_{L3B} is s times a capacitance matrix. This matrix is calculated by populating the non-zero elements and adding its transpose (the pattern of non-zero elements to be updated later).
Y_{L3B}=s·(C_{3B}+C_{3B}^{T})

Summary of the Calibration Algorithm:

All calibration operations discussed are divided into two software processes: static calibration and dynamic calibration. Static calibration is a “long term” calibration and may be executed at “Session Test” at the beginning of treatment for each patient. Dynamic calibration is interleaved with localization measurements.

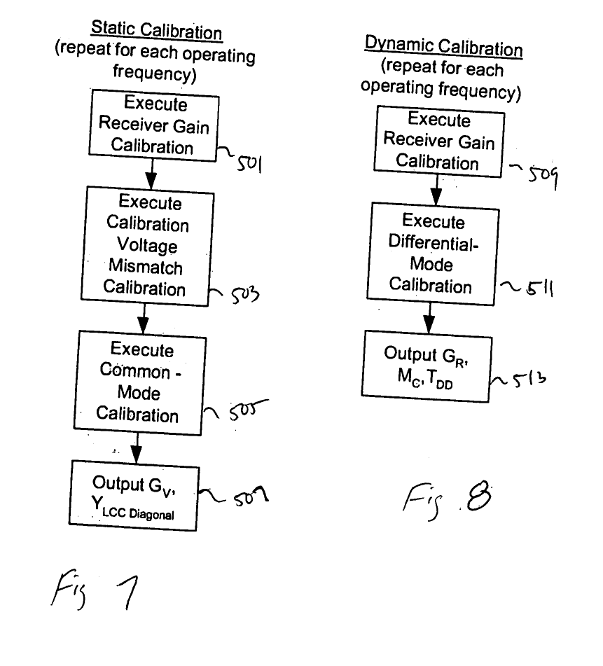

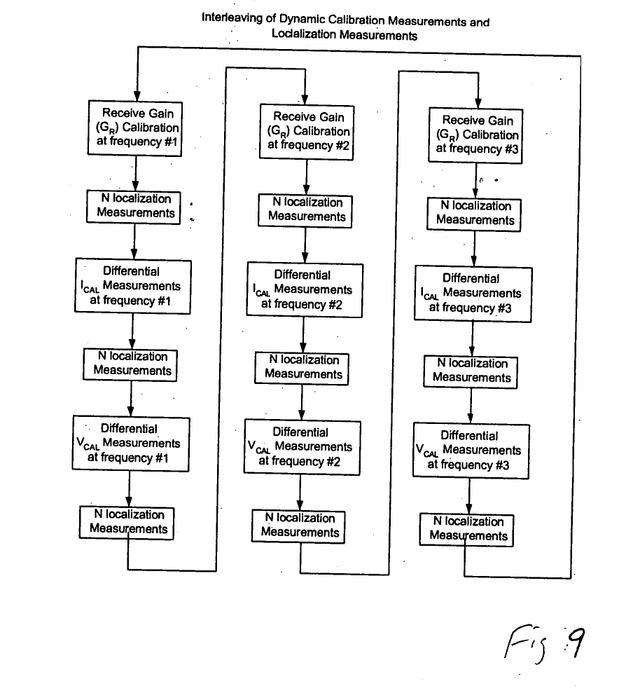

FIGS. 7 and 8 indicate the processes involved with each. As seen in FIG. 7, at box 501, a receiver gain calibration is performed. Then at box 503, a voltage mismatch calibration is performed. At box 505, the common mode calibration is performed. Once these calibration steps have been performed, the Gᵥ and Y_{LCCdiag} matrices can be calculated and output at box 507.

As seen in FIG. 8, at box 509, a receiver gain calibration is performed. Then at box 511, a differential mode calibration is performed. Once these calibration steps have been performed, the G_{R}, M_{c} and T_{DD} matrices can be calculated and output at box 507.

FIG. 9 describes how dynamic calibration measurements are interleaved with localization measurements. Note that in FIG. 9, the iteration parameter N will be typically around 4. Each localization measurement requires roughly 100 msecs. Each calibration block requires roughly 40 msec. Executing the full dynamic calibration cycle thus will take roughly 4 seconds. Note that three frequencies for the markers are present for the typical case where three markers (of presumably differing resonant frequencies) are implanted.

The generation of the various correction matrices described above can be more broadly stated as the generation of “noise corrections” or “corrections to a sensed signal”.

Receiver Gain (G_{R}) Calibration

The objective of Receiver Gain Cal is to back out gain errors associated with the higher gain typically used in localization (G_{R}⁻¹). Calibration is performed at the low-gain setting. If localization is performed with the low-gain setting, no correction is needed. If localization is performed with the high-gain setting, the measured S-vectors should be left-multiplied by G_{R} prior to any other processing.

The following steps are performed in receive gain calibration 501 and 509:
1) Configure receiver front-end for high-gain with inputs selected to V_CM
2) Measure S_HIGH complex values with cal-kernel, NMSI=1
3) Configure receiver front-end for low-gain with inputs selected to V_CM
4) Measure S_LOW complex values with cal-kernel, NMSI=1
5) Form gr_inv, a 32×1 complex vector by taking the element-by element ratio of S_HIGH over S_LOW.
6) Calculate G_{R}=(diag{gr_inv})⁻¹
7) Output G_{R}

Calibration Voltage Mismatch (G_{V}) Calibration:

The objective of this process is to determine the channel-channel gain mismatches present in the calibration voltage sources.

The following steps are performed to determine G_{V}:
1. Configure receiver in low gain and differential reception
2. Excite Cal Current sources one at a time differentially. The 32 sets of measured S vectors form the matrix S_VCAL_{DD}.
3. Excite Cal Voltage sources one at a time differentially. The 32 sets of measured S vectors form the matrix S_iCAL_{DD}.
4. Calculate T_{DD}, Y_{LDDMeasured}, as follows:

$${T}_{DD} = \frac{1}{{R}_{REF}} {( {S}_{{ICAL}_{DD}} )}^{- 1}$$

$${Y}_{LDDMeasured} = - \frac{1}{{K}_{DIV}} {T}_{DD} \cdot {S}_{{VCAL}_{DD}}$$
5. Form the matrix A from the elements of Y_{LDDMeasured} and construct the vector b as described in section 4.
6. Obtain the correction gain vector g and matrix G_{V} as follows:
   g=(A^{H}A)⁻¹A^{H}b
   G_{V}=diag{g}
7. Output G_{V}.

Common-Mode Calibration:

The objective of this process is to measure the diagonal terms of the common-mode block of Y_{L}, designated as Y_{LCCdiagonal}.

The following steps are performed to determine Y_{LCCDiagonal}:
1) Configure receiver in low gain and single-ended receive (+terminal)
2) Excite Cal Current sources one at a time in common-mode. The 32 sets of measured S vectors form the matrix S_ICAL_{C+}.
3) Repeat steps 1 and 2 for −terminal to collect S_ICAL_{C-}.
4) Excite Cal Voltage sources one at a time in common mode receiving single-ended both + and − terminals. The two 32×32 sets of measured S vectors form the matrices S_VCAL_{C+} and S_VCAL_{C-}.
5) Calculate T_{CC} and use G_{V} (see below) to calculate Y_{LCCDiagonal} as follows:

$${T}_{CC} = \frac{1}{{R}_{REF}} {( {S}_{{ICAL}_{C +}} - {S}_{{ICAL}_{C -}} )}^{- 1}$$

$${Y}_{LCC} = - \frac{1}{{K}_{DIV}} {T}_{CC} \cdot ( {S}_{{VCAL}_{C +}} - {S}_{{VCAL}_{C -}} ) \cdot {G}_{V}$$

Zero out all non-diagonal elements of Y_{LCC} to form Y_{LCCDiagonal}
6) Output Y_{LCCDiagonal}.

Differential-Mode Calibration

The two objectives of differential-mode calibration are to:
Frequently update the differential block of the T matrix relating measured S values to short circuit current at the preamp inputs
Frequently update the transfer function from V_{EXT} to differential short circuit current at the preamp inputs (ie M_{C}⁻¹).

The following steps are performed for Differential Cal:
1. Configure receiver in low gain and differential reception
2. Excite Cal Current sources one at a time differentially. The 32 sets of measured S vectors form the matrix S_ICAL_{DD}.
3. Excite Cal Voltage sources one at a time differentially. The 32 sets of measured S vectors form the matrix S_VCAL_{DD}.
4. Calculate T_{DD}, Y_{LDDMeasured}, and M_{C} as follows:

$${T}_{DD} = \frac{1}{{R}_{REF}} {( {S}_{{ICAL}_{DD}} )}^{- 1}$$

$${Y}_{LDDMeasured} = - \frac{1}{{K}_{DIV}} {T}_{DD} \cdot {S}_{{VCAL}_{DD}}$$

$${Y}_{LDDCorrected} = {Y}_{LDDMeasured} \cdot {G}_{V} - {Y}_{LCCDiagonal} - {Y}_{L3A} - {Y}_{L3B}$$

$${M}_{C} = {( {Y}_{LDDCorrected} )}^{- 1}$$
5. Output T_{DD} and M_{C}.

Application of Calibration Results to raw S vectors for Localization:
S_{CORRECTED}=M_{C}·T_{DD}·G_{R}·S_{RAW}
Dead Reckoning Application

The above description contemplates that application of corrections to a sensed signal to the sensed signals from a specific sensing array. In other words, each sensing array may be calibrated individually and in real time. However, for some applications, it may be that the noise corrections are very nearly the same for all sensing arrays that are manufactured to a particular specification or in a common batch. In such a situation, a “dead reckoning” method may be used wherein the calibration technique is performed on a selected sensing array. The noise corrections developed from that calibration on the selected sensing array is then applied to all of the sensing array in a defined batch. In such a manner, the other sensing arrays need not perform the calibration.

Conclusion

Unless the context clearly requires otherwise, throughout the description and the claims, the words “comprise,” “comprising,” and the like are to be construed in an inclusive sense as opposed to an exclusive or exhaustive sense, that is to say, in the sense of “including, but not limited to.” Words using the singular or plural number also include the plural or singular number, respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list, and any combination of the items in the list.

The above detailed descriptions of embodiments of the invention are not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, an array of hexagonally shaped sense coils may be formed on a planar array curved along at least one line to form a concave structure. Alternatively, the arrangement of coils on the panel may form patterns besides the “cross” pattern shown in FIGS. 3A and 3B. The coils may be arranged on two or more panels or substrates, rather than the single panel described herein. The teachings of the invention provided herein can be applied to other systems, not necessarily the system employing wireless, implantable resonating targets described in detail herein. These and other changes can be made to the invention in light of the detailed description.

The elements and acts of the various embodiments described above can be combined to provide further embodiments. All of the above U.S. patents and applications and other references are incorporated herein by reference. Aspects of the invention can be modified, if necessary, to employ the systems, functions and concepts of the various references described above to provide yet further embodiments of the invention.

These and other changes can be made to the invention in light of the above detailed description. In general, the terms used in the following claims should not be construed to limit the invention to the specific embodiments disclosed in the specification, unless the above detailed description explicitly defines such terms. Accordingly, the actual scope of the invention encompasses the disclosed embodiments and all equivalent ways of practicing or implementing the invention under the claims.

One skilled in the art will appreciate that although specific embodiments of the location system have been described herein for purposes of illustration, various modifications may be made without deviating from the spirit and scope of the invention. Accordingly, the invention is not limited except by the appended claims.

1. A method for calibrating a sensing array used for marker localization, the sensing array including a plurality of sensing elements, the method comprising:
applying an excitation to at least one of said plurality of sensing elements of said sensing array used for marker localization;
analyzing the output of some or all of said plurality of sensing elements resulting from said excitation;
repeating said excitation and analyzing process for each of said plurality of sensing elements;
determining corrections to a sensed signal based upon said analyzed outputs of said plurality of sensing elements.

2. The method of claim 1 wherein each sensing element has a corresponding preamplifier.

3. The method of claim 1 wherein said corrections are applied to the outputs of said sensing array during marker localization.

4. The method of claim 2 wherein said preamplifier is a differential amplifier having first and second amplification elements, wherein said excitation of one of said plurality of sensing elements includes applying an exciting voltage sequentially to said first and second amplification elements.

5. The method of claim 2 wherein said preamplifier is a differential amplifier having first and second amplification elements, wherein said excitation of one of said plurality of sensing elements includes applying an exciting current sequentially to said first and second amplification elements.

6. The method of claim 1 wherein said excitation is applied to less than all of the sense coils in said sensing array.

7. The method of claim 1 wherein said excitation is a voltage to said sensing element.

8. The method of claim 1 wherein said excitation is a current to said sensing element.

9. The method of claim 7 wherein said voltage is a sinusoidal wave.

10. The method of claim 8 wherein said current is a sinusoidal wave.

11. The method of claim 1 further wherein the calibrating method is interleaved between marker localization operations.

12. A method for calibrating a sensing array used for marker localization, the sensing array including a plurality of sensing elements, said plurality of sensing elements including a calibration subset selected from said plurality of sensing elements, comprising:
applying an excitation to one of said plurality of sensing elements in said calibration subset of said sensing array used for marker localization;
analyzing the output of some or all of said plurality of sensing elements resulting from said excitation;
repeating said excitation and analyzing process for each of the sensing elements in said calibration subset; and
determining corrections to a sensed signal based upon said analyzed outputs.

13. The method of claim 12 wherein each sensing element has a corresponding preamplifier.

14. The method of claim 12 wherein said corrections are applied to the outputs of said sensing array during marker localization.

15. The method of claim 13 wherein said preamplifier is a differential amplifier having first and second amplification elements, wherein said excitation of at least one of said plurality of sensing elements includes applying an exciting voltage sequentially to said first and second amplification elements.

16. The method of claim 13 wherein said preamplifier is a differential amplifier having first and second amplification elements, wherein said excitation of at least one of said plurality of sensing elements includes applying an exciting current sequentially to said first and second amplification elements.

17. The method of claim 12 wherein said excitation is a sinusoidal voltage to said sensing element.

18. The method of claim 2 wherein said excitation is a sinusoidal current to said sensing element.

19. The method of claim 2 wherein an excitation is applied to more than one of said plurality of sensing elements simultaneously.

20. The method of claim 2 further wherein the calibrating method is interleaved between marker localization operations.

21. A method for calibrating a sensing array used for marker localization, the sensing array including a plurality of sensing elements, said plurality of sensing elements including a calibration subset selected from said plurality of sensing elements, comprising:
applying a voltage excitation to one of said plurality of sensing elements in said calibration subset of said sensing array used for marker localization;
analyzing the output of some or all of said plurality of sensing elements resulting from said excitation;
repeating said voltage excitation and analyzing process for each of the sensing elements in said calibration subset; and
determining corrections to a sensed signal based upon said analyzed outputs.

22. The method of claim 21 wherein each sensing element has a corresponding preamplifier.

23. The method of claim 21 wherein said corrections are applied to the outputs of said sensing array during marker localization.

24. The method of claim 21 wherein said preamplifier is a differential amplifier having first and second amplification elements, wherein said voltage excitation of at least one of said plurality of sensing elements includes applying an exciting voltage sequentially to said first and second amplification elements.

25. The method of claim 21 wherein said voltage excitation is a sinusoidal wave.

26. The method of claim 21 wherein said calibration subset includes all of said plurality of sensing elements.

27. The method of claim 21 wherein a voltage excitation is applied to more than one of said plurality of sensing elements simultaneously.

28. The method of claim 21 further wherein the calibrating method is interleaved between marker localization operations.

29. A apparatus for calibrating a sensing array used for marker localization, the sensing array including a plurality of sensing elements, said plurality of sensing elements including a calibration subset selected from said plurality of sensing elements, comprising:
a source for applying an excitation to one of said plurality of sensing elements in said calibration subset of said sensing array used for marker localization;
means for analyzing the output of some or all of said plurality of sensing elements resulting from said excitation;
means for repeating said excitation and analyzing process for each of the sensing elements in said calibration subset; and
means for determining corrections to a sensed signal based upon said analyzed outputs.

30. The apparatus of claim 29 wherein each sensing element has a corresponding preamplifier.

31. The apparatus of claim 29 wherein said corrections are applied to the outputs of said sensing array during marker localization.

32. The apparatus of claim 30 wherein said preamplifier is a differential amplifier having first and second amplification elements, wherein said excitation of at least one of said plurality of sensing elements includes applying an exciting voltage sequentially to said first and second amplification elements.

33. The apparatus of claim 30 wherein said preamplifier is a differential amplifier having first and second amplification elements, wherein said excitation of at least one of said plurality of sensing elements includes applying an exciting current sequentially to said first and second amplification elements.

34. The apparatus of claim 29 wherein said excitation is a sinusoidal voltage to said sensing element.

35. The apparatus of claim 29 wherein said excitation is a sinusoidal current to said sensing element.

36. The apparatus of claim 29 wherein an excitation is applied to more than one of said plurality of sensing elements simultaneously.

37. The apparatus of claim 29 further wherein the calibrating method is interleaved between marker localization operations.

38. A method for calibrating multiple sensing arrays, each sensing array used for marker localization, the sensing array including a plurality of sensing elements, said plurality of sensing elements including a calibration subset selected from said plurality of sensing elements, comprising:
for one of said sensing arrays:
(a) applying an excitation to one of said plurality of sensing elements in said calibration subset of said sensing array used for marker localization;
(b) analyzing the output of some or all of said plurality of sensing elements resulting from said excitation;
(c) repeating said excitation and analyzing process for each of the sensing elements in said calibration subset; and
(d) determining noise corrections based upon said analyzed outputs; and
using said noise corrections determined for said one of said sensing arrays in the other sensing arrays.

39. The method of claim 38 wherein said excitation is a sinusoidal voltage to said sensing element.

40. The method of claim 38 wherein said excitation is a sinusoidal current to said sensing element.

41. The method of claim 38 wherein an excitation is applied to more than one of said plurality of sensing elements simultaneously.

42. A method for calibrating a sensing array used for marker localization, the sensing array including a plurality of sensing elements, said plurality of sensing elements including a calibration subset selected from said plurality of sensing elements, comprising:
applying a current excitation to one of said plurality of sensing elements in said calibration subset of said sensing array used for marker localization;
analyzing the output of some or all of said plurality of sensing elements resulting from said excitation;
repeating said current excitation and analyzing process for each of the sensing elements in said calibration subset; and
determining corrections to a sensed signal based upon said analyzed outputs.

43. The method of claim 42 wherein each sensing element has a corresponding preamplifier.

44. The method of claim 42 wherein said corrections are applied to the outputs of said sensing array during marker localization.

45. The method of claim 43 wherein said preamplifier is a differential amplifier having first and second amplification elements, wherein said current excitation of at least one of said plurality of sensing elements includes applying an exciting current sequentially to said first and second amplification elements.

46. The method of claim 42 wherein said current excitation is a sinusoidal wave.

47. The method of claim 42 wherein said calibration subset includes all of said plurality of sensing elements.

48. The method of claim 42 wherein a current excitation is applied to more than one of said plurality of sensing elements simultaneously.

49. The method of claim 42 further wherein the calibrating method is interleaved between marker localization operations.
